(19) United States
(12) Patent Application Publication
(10) Pub. No.: US 2025/0329426 A1
(43) Pub. Date: Oct. 23, 2025

(54) TRANSFER DEVICE FOR TRANSFERING AT LEAST ONE FILE STORED ON A PROGRAMMING DEVICE TO A REMOTE PLATFORM

(71) Applicant: IMPLICITY, Paris (FR)

(72) Inventor: David PERLMUTTER, Paris (FR)

(73) Assignee: IMPLICITY, Paris (FR)

(21) Appl. No.: 18/637,548

(22) Filed: Apr. 17, 2024

(51) Int. Cl. G16H 15/00; G06F 13/10; H04L 67/06

(52) U.S. Cl. CPC G16H 15/00; G06F 13/105; H04L 67/06

(57) ABSTRACT

A transfer device and associated method for transferring at least one file stored on a programming device to a remote platform. The programming device is configured to generate at least one file from data received from a medical device. The transfer device streamlines the process of transferring files from a programming device to a remote platform.

### FIELD

The present invention relates to the technical field of medical data transmission. More precisely, the invention pertains to a transfer device and associated method for transferring at least one file stored on a programming device to a remote platform.

### BACKGROUND

In the domain of monitoring patients using electronic medical devices (e.g. medical devices intended to be implanted in patient's body such as for instance Cardiac Implantable Electronic Devices (CIED) like pacemakers or defibrillators, or medical devices intended to be inserted under the skin like Inserted Loop Recorders, or external wearable devices like external Electrocardiogram monitors (Holters)), the management of the medical device plays a pivotal role in ensuring the well-being of patients. Central to this management is the need to regularly review and adjust settings of the medical device, a task typically performed by physicians, during in person clinic visit, using specialized programming computers.

In addition to their programming features, these programming computers can generate exportable data in the form of files. These files encompass essential information to be attached to the physician consultation report, such as PDF reports including the main settings of the medical device. They may also contain a comprehensive overview of measurements taken by the medical device, and the history of alerts since the last connection to the programming computer. These reports contain both textual data and graphical representations illustrating the evolution of values over time. The files may also comprise the medical device setting data files, that come in various formats specific to each manufacturer and are not intended for printing. These setting data files provide detailed information on how the device has been configured by the physician (all internal settings the physician can use to configure the device according to the patient's condition).

Physicians frequently collaborate with remote platforms, such as cloud-based telemonitoring systems, to facilitate continuous monitoring and management of patients equipped with various types of medical devices. These platforms provide a centralized hub for aggregating and processing data into patients' files, that physicians can access for instance when an alert is received. Notably, the data generated during the in-person consultation should be included in the patient's file, because it contains all medical device settings adjustments made by the physician during the consultation that should be reported in the medical report created by the physician during the in-person visit.

However, the process of transferring these files, from the programming computers to the remote platforms, has traditionally been cumbersome and complex. Physicians often find themselves navigating through intricate procedures involving multiple steps and manual operations (e.g. for instance: exporting the files to a USB key, inserting the USB key into a personal computer, uploading the files to the cloud or printing them, moving the files to the appropriate patient's file), all of which consume valuable time and resources when the physician only has a short time between two patients visits.

This complexity not only presents practical challenges but also creates potential barriers to timely and efficient patient care. With the increasing emphasis on remote patient monitoring and data-driven healthcare delivery, there arises a pressing need for a simpler and more streamlined method of transferring files generated by programming computers to remote platforms.

### SUMMARY

This invention thus relates to a transfer device for transferring at least one file stored on a programming device to a remote platform, said programming device being configured to generate said at least one file from at least one data received from a medical device, said transfer device comprising:
at least one input interface configured to receive said at least one file from said programming device,
a memory comprising a memory space configured to receive said at least one file from said at least one input interface,
at least one processor configured to:
   detect when a connection is established between said transfer device and said programming device,
   detect when said at least one file is written into said memory space after said connection is established,
at least one output interface configured to send said at least one file to said remote platform, wherein said at least one processor is further configured to transmit said at least one file, after detection that said at least one file has been written into said memory space, from said memory space to said remote platform via said at least one output interface.

Advantageously, the present invention provides a transfer device designed to streamline the process of transferring files from a programming device to a remote platform. By automatically detecting when files are written into the memory space, the transfer device simplifies data transfer, optimizing resource utilization and ultimately enabling fast medical reports generation. With reliable file transmission capabilities and minimal user intervention required, physicians can initiate file transfer requests with ease, enhancing efficiency and maintaining the integrity of patient data throughout the process. Moreover, physicians can finalize the medical report (with all the necessary data) during the short time of the consultation (less than 15 minutes in average) so that physicians no longer have to perform any administrative task once the patient leaves the consultation room.

According to other advantageous aspects of the invention, the transfer device comprises one or more of the features described in the following embodiments, taken alone or in any possible combination.

According to one embodiment, said transfer device is configured to emulate a behavior of a Universal Serial Bus (USB) storage device so that when said connection is established between said transfer device and said programming device, said programming device authorizes said writing of said at least one file into said memory space.

In practice, writing and reading authorizations are provided temporarily. The authorization may be withdrawn while the transfer device transfers and delete the files from the memory space. Afterwards, writing and reading authorizations may be further granted to allow new files to be transferred.

According to one embodiment, when said transfer device is configured to emulate a behavior of a Universal Serial Bus (USB) storage, said memory space is a shared memory space and said at least one processor is configured to share said shared memory space with the programming device.

By mimicking the behavior of a USB storage device, the transfer device is compatible with all programming devices equipped with as USB port.

According to one embodiment, said transfer device is configured to emulate a behavior of a printer so that when said connection is established between said transfer device and said programming device, said programming device authorizes said writing of said at least one file into said memory space.

In practice, writing and reading authorizations are provided temporarily. The authorization may be withdrawn while the transfer device transfers and delete the files from the memory space. Afterwards, writing and reading authorizations may be further granted to allow new files to be transferred.

The printer according to the invention is a printer that may be connected with at least one of the following protocols: USB, or WiFi, or Bluetooth or Ethernet.

Notably, by adhering to established Bluetooth communication standards, the transfer device ensures seamless compatibility and interoperability with all programming devices commonly used in medical settings that can establish a Bluetooth connection. This approach enables efficient and reliable data transfer, as Bluetooth protocols facilitate secure communication between the programming device and the memory of the transfer device.

According to one embodiment, when transmitting said at least one file from said memory space to said remote platform fails, said at least one processor is configured to initiate again at least one attempt of transmitting said at least one file from said memory space to said remote platform.

By automatically initiating additional transmission attempts from the memory space to the remote platform upon a failed transmission, the transfer device is robust to short network interruption, reducing the need of manual retry in case of network failure.

Additionally, the processor may be further configured to send a warning signal to the user (for instance a visual signal by switching on a light) to inform him of transmission failure.

According to one embodiment, when a number of failed attempts of transmitting said at least one file from said memory space to said remote platform exceeds a threshold, said at least one processor is configured to delete said at least one file from said memory space.

According to the invention, the threshold may be a number of failed attempts or a predetermined period of time.

In other words, when the threshold is exceeded, the transfer device automatically initiates the deletion of files from the memory space, preventing the accumulation of unsuccessful transmission files. This proactive approach optimizes resource utilization by freeing up storage space and minimizing clutter. Additionally, the automatic deletion plays a crucial role in safeguarding patient privacy. By promptly removing files that have not been successfully transmitted, the transfer device ensures that patients' private medical data remains protected from any potential information leaks or unauthorized access. Additionally, the at least one processor may be further configured to display an error status to the user (e.g. for instance the physician).

According to one embodiment, the at least one processor is further configured to delete said at least one file from said memory space after said at least one file has been correctly transmitted to said remote platform.

By automatically deleting files from the memory space after successful transmission to the remote platform, the transfer device ensures data security by reducing the likelihood of unintended access or exposure to sensitive information.

According to one embodiment, said transfer device is configured to establish a connection with multiple programming devices.

According to one embodiment, said at least one processor is configured to select one programming device among the multiple programming devices from which said at least one input interface is configured to receive said at least one file.

This enhances versatility and flexibility in medical settings where multiple programming devices may be utilized. By accommodating various programming devices, the transfer device ensures seamless integration into diverse healthcare environments without the need for additional hardware or software modifications. Overall, this embodiment enhances operational efficiency, workflow flexibility, and interoperability, thereby improving healthcare delivery in medical settings.

According to one embodiment, said at least one processor is further configured, before transmission of said at least one file to said remote platform, to send a transmission request to a transmitter service of said remote platform and to notify a transmission success to said transmitter service.

According to one embodiment, said at least one processor is configured to:
request a transfer of said at least one file to said remote platform,
receive a single usage URL from said remote platform,
transmit said at least one file using said received single usage URL, and
notify a transmission success after said at least one file has been successfully transmitted.

This offers streamlined data transfer processes and minimizes unnecessary data transfers. By having two different API (one in the transfer device and one in the remote platform, the burden of hosting an API is distributed between the transfer device and the remote platform. Upon receiving transmission validation, the transfer device confirms the successful transfer, providing assurance that critical patient data has been securely transmitted and triggers further steps of file processing by the remote platform. Importantly, this method minimizes data transfer by only sending the file once.

The present invention further relates to a computer-implemented method for transferring at least one file from a programming device to a remote platform, said programming device being configured to generate said at least one file from at least one data received from a medical device, said method being implemented with the transfer device described above, said method comprising the steps of:
establishing a connection between said transfer device and said programming device,
detecting when said at least one file is written into said memory space,
transmitting said at least one file from said memory space to said remote platform.

According to other advantageous aspects of the invention, the method comprises one or more of the features described in the following embodiments, taken alone or in any possible combination.

According to one embodiment, when the transfer device is configured to emulate the behavior of a USB storage device, the method further comprises a step of sharing with said programming device the shared memory space.

According to one embodiment, the method further comprises notifying to a user success or failure of said transmission.

The notification may be made by sending at least one signal to the user (for instance a visual signal or a sound signal).

According to one embodiment, the method further comprises storing said at least one file into a storage space of said remote platform.

According to one embodiment, the method further comprises processing, by a file parser service of said remote platform, said at least one data comprised in said at least one file.

In practice, such processing may be triggered when file transmission has been validated.

This enables the extraction of structured data from unstructured files, enhancing the efficiency and accuracy of data analysis and interpretation. By utilizing the file parser service, the remote platform can automatically interpret and extract meaningful information from the files, eliminating the need for manual data entry or interpretation by physicians. This automation streamlines workflows, saving time and reducing the potential for errors associated with manual data processing (e.g. errors in files classification by physicians). Moreover, the file parser service enhances data integrity by ensuring consistent and standardized data extraction, leading to more reliable insights and decision-making in healthcare settings. Additionally, by converting unstructured data into structured formats, the file parser service facilitates seamless integration with other healthcare systems and applications, supporting interoperability and data exchange.

According to one embodiment, said processing comprises extracting at least one data of interest from said at least one file and automatically generating a medical report based on at least said data of interest.

The medical report may be generated based on the data of interest and other data already present on the remote platform. Such data may be extracted from files previously received by the remote platform, for instance via the transfer device.

According to one embodiment, when said data of interest comprise new data generated by said medical device, said processing comprises extracting said new data from said at least one file and automatically updating said medical report based at least on said new data.

The automatic generation of medical reports significantly reduces the time and effort required by physicians to fill out medical reports. Furthermore, by utilizing templates of reports, the embodiment ensures consistency and standardization in report formatting and content, enhancing the quality and reliability of the generated reports.

According to one embodiment, the method further comprises reviewing and validating, by a physician, the medical report via said remote platform.

According to other embodiments, the method may further comprise editing and/or signing the medical report.

### Definitions

In the present invention, the following terms have the following meanings:

The terms “adapted” and “configured” are used in the present disclosure as broadly encompassing initial configuration, later adaptation or complementation of the present devices, or any combination thereof alike, whether effected through material or software means (including firmware).

The term “processor” should not be construed to be restricted to hardware capable of executing software, and refers in a general way to a processing device, which can for example include a computer, a microprocessor, an integrated circuit, or a programmable logic device (PLD). The processor may also encompass one or more Graphics Processing Units (GPU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.

### BRIEF DESCRIPTION OF THE DRAWINGS

The present disclosure will be better understood, and other specific features and advantages will emerge upon reading the following description of particular and non-restrictive illustrative embodiments, the description making reference to the annexed drawings wherein:

FIG. 1 is a bloc diagram illustrating the transfer of files from the programming device to the remote platform using the transfer device according to one embodiment of the invention;

FIG. 2 is a bloc diagram showing elements comprised in the transfer device from FIG. 1;

FIG. 3 is a bloc diagram showing elements comprised in the remote platform from FIG. 1;

FIG. 4 is a perspective view of the transfer device from FIG. 1;

FIG. 5 is a bloc diagram showing steps of a method implemented by the transfer device from FIG. 1;

FIG. 6 is a bloc diagram showing a first embodiment of connection between a programming device according to the invention and a transfer device according to the invention;

FIG. 7 is a bloc diagram showing a second embodiment of connection between a programming device according to the invention and a transfer device according to the invention;

FIG. 8 is a bloc diagram showing a third embodiment of connection between a programming device according to the invention and a transfer device according to the invention.

On the figures, the drawings are not to scale, and identical or similar elements are designated by the same references.

### DETAILED DESCRIPTION

The present description illustrates the principles of the present disclosure. It will thus be appreciated that those skilled in the art will be able to devise various arrangements that, although not explicitly described or shown herein, embody the principles of the disclosure and are included within its scope.

All examples and conditional language recited herein are intended for educational purposes to aid the reader in understanding the principles of the disclosure and the concepts contributed by the inventor to furthering the art, and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that the block diagrams presented herein may represent conceptual views of illustrative circuitry embodying the principles of the disclosure. Similarly, it will be appreciated that any flow charts, flow diagrams, and the like represent various processes which may be substantially represented in computer readable media and so executed by a computer or processor, whether or not such computer or processor is explicitly shown.

The functions of the various elements shown in the figures may be provided through the use of dedicated hardware as well as hardware capable of executing software in association with appropriate software. When provided by a processor, the functions may be provided by a single dedicated processor, a single processor, or a plurality of individual processors, some of which may be shared.

It should be understood that the elements shown in the figures may be implemented in various forms of hardware, software or combinations thereof. Preferably, these elements are implemented in a combination of hardware and software on one or more appropriately programmed general-purpose devices, which may include a processor, memory and input/output interfaces.

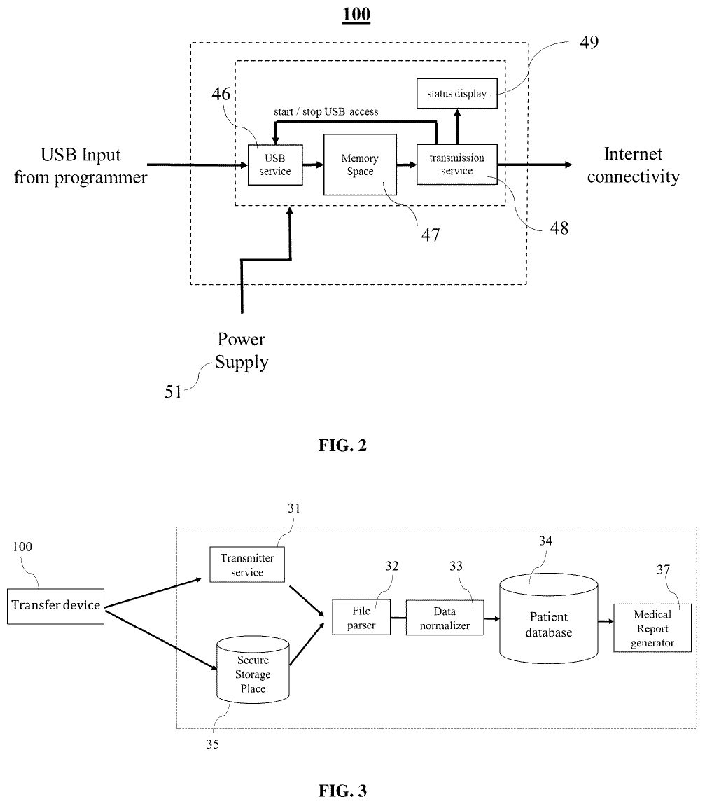

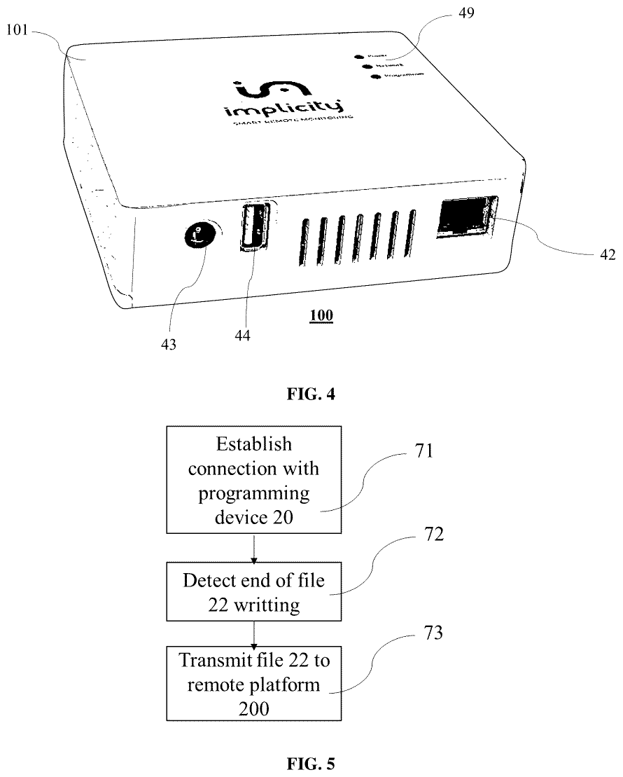

The present disclosure will be described in reference to a particular functional embodiment of a transfer device 100 as illustrated on FIG. 2.

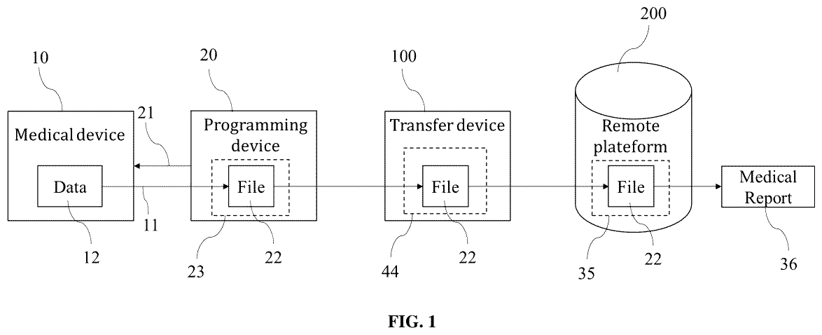

The transfer device 100 is configured to enable the transfer of files 22 stored on a programming device 20 toward a remote platform 200, such as illustrated on FIG. 1.

The programming device 20 is designed for interfacing with medical devices 10.

According to the invention, a medical device 10 refers to a wearable or implantable medical device worn by a subject. Such medical device 10 may for instance be a Cardiac Implantable Electronic Devices (CIED) like pacemakers.

The programming device 20 may be a computer device, a workstation, a laptop, a portable handheld unit such as a tablet or a smartphone, or an external unit configured to be positioned directly on the skin of the patient to get as close as possible from the medical device 10.

The programming device 20 may comprise at least one of the following elements, connected to each other by a bus of addresses and data that also transports a clock signal:
a microprocessor (or CPU);
a graphics card comprising several Graphical Processing Units (or GPUs) and a Graphical Random Access Memory (GRAM);
a non-volatile memory of ROM type and/or Hard disk drive (HDD) type and/or Flash type and/or NVRAM type;
a RAM; and
a power supply.

It is noted that the memories (GRAM, ROM, RAM) can designate in each of the memories mentioned, a memory zone of low capacity (some binary data) as well as a memory zone of large capacity (enabling a whole program to be stored or all or part of the data representative of data calculated or to be displayed).

The programming device 20 may also comprises a display device of display screen type to allows visualization of data relative to the medical device 10. According to a variant, the display device may be external to the programming device 20 and connected thereto by a cable or wirelessly. The programming device 20, for example through the graphics card, comprises an interface for transmission or connection adapted to transmit a display signal to an external display means such as for example an LCD or plasma screen or a video-projector.

The programming device may further comprise at least one interface unit configured to allow a user, such as the physician, to interact with the programming device 20, for example to enter commands. For that purpose, the programming device 20 may comprise Input devices such as for example at least one of a keyboard, a mouse, a trackball, a webcam and a vocal recognition.

The programming device 20 may be configured to establish connection with the medical device 10 and the transfer device 100, through wired connection such as USB, Ethernet, or other data cables, or through wireless connection such as Bluetooth, Wi-Fi, Infrared, and other suitable communication protocols or technologies based on the specific requirements and constraints of the medical device 10 and the environment in which they operate. To that end, the programming device 20 may comprise at least one of a USB port and an Ethernet port.

Through this connection, the programming device 20 is configured to send 21 data 12 to the medical device 10 and to retrieve 11 data 12 from the device 20.

The data 12 sent 21 to the medical device 10 may take the form of management data that allows for various actions such as configuring the settings of the medical device 10, updating its firmware, performing diagnostic functions, adjusting parameters, initiating self-tests, calibrating sensors, verifying integrity of the medical device 10 and managing security features.

The data 12 retrieved 11 from the medical device 10 may take the form of telemetric data, such as event logs, performance metrics, sensor readings or other relevant metrics enabling physicians to monitor patient health and device performance, even from a remote location. Such data may include text, images, audio, or video.

The data 12 retrieved by the programming device 20 from the medical device 10 may be processed by the programming device 20 to generate files 22 (e.g. digital container used for storing the data 12 with a given organization and/or structure and a specific format, allowing for easy access, retrieval, and manipulation by software applications and users). Each file 22 is identified by a unique name and may have associated attributes such as size, creation date, and file type. Alternatively, at least one of the files 22 may be generated directly by the medical device 10 and retrieved 11 and stored by the programming device 20 with no further processing.

For instance, the management data may be organized as program instruction files, for execution by the medical device 10, configuration files defining operational parameters for the medical device 10, security files containing cryptographic keys, access control lists, or authentication protocols to ensure secure communication and data protection between the medical device 10 and the programming device 20. These types of files typically have different formats, specific to each manufacturer and not intended to be printed, such as the raw format.

The telemetric data may be organized into alert files, measurement files, graphical representations files, or diagnostic files typically containing error messages, warnings, system events, device states, and other diagnostic information generated by the medical device 10 itself or by accompanying software used for troubleshooting, debugging, and monitoring purposes. These types of files may be of a printable format such as the PDF format.

The files 22 are stored on the at least one memory 23 of the programming device 20. They may be opened, edited, saved, copied, and deleted using appropriate software tools and applications and using the display screen for visualization of the above-mentioned actions.

The remote platform 200 provides a centralized hub for aggregating and processing data relative to one patient or multiple patients, that physicians can access and analyze remotely. The remote platform 200 may also provide a way for physicians to generate and edit medical reports 36.

The remote platform 200 is configured to receive 41 the files 22 generated or retrieved by the programming device 20, via the transfer device 100.

To that end, as illustrated on FIG. 3, the remote platform 200 may comprise a transmitter service 31, which includes the software or protocols responsible for receiving and sending data packets from/to the transfer device 100. The remote platform 200 may for instance utilize Application Programming Interfaces (APIs) to communicate with the transfer device 100. The transmitter service 31 may notably be configured to act as the gatekeeper for authorizing data transmissions within the remote platform 200.

The transmitter service 31 may be configured to receive transmission requests from transfer devices 100. A transmission request is a formal inquiry or solicitation made by the transfer device 100 to gain authorization or permission to access certain resources, services, or data within the remote platform 200. For instance, the transmission request may be transmitted using HTTPS (HTTP Secure) requests or TCP/IP (Transmission Control Protocol/Internet Protocol).

Other file transfer protocols may be used such as FHIR (Fast Healthcare Interoperability Resources), SFTP (Secure File Transfer Protocol), MQTT (Message Queuing Telemetry Transport), HL7 (Health Level Seven), MLLP (Minimal Lower Layer Protocol), SCP (Secure Copy Protocol).

Upon receiving a request, the transmitter service 31 validates the authenticity and integrity of the request, ensuring that it complies with security protocols and access controls. Once validated, the transmitter service 31 may generate a unique URL. The URL may be unique and/or time-limited and/or configured for a single usage. The transmitter service 31 may be configured to send the URL back to the requesting transfer device 100. This URL serves as a secure authentication token, granting the requester permission to initiate the transmission process. By employing this method, the transmitter service 31 enhances the security of data transmissions, mitigating the risk of unauthorized access or data breaches while facilitating seamless and efficient file transfers within the remote platform 200.

The transmitter service 31 may further be configured to run an anti-virus on the files 22.

The remote platform 200 may further comprise a secure storage space 35 configured to store the files 22, received by the transfer device 100, ensuring data integrity and compliance with privacy regulations. The secure storage space 35 may refer to a server or a set of servers dedicated to storing and managing the files 22. These servers are equipped with storage hardware, such as hard disk drives (HDDs) or solid-state drives (SSDs), and run software applications or services responsible for organizing, storing, retrieving, and securing data.

Upon reception of the files 22, the remote platform 200 may be configured to send an acknowledgment message to the transfer device 100. This acknowledgment message serves to confirm to the transfer device 100 that the files 22 have been successfully received by the remote platform 200 without errors. In networked systems, such as those utilizing TCP/IP (Transmission Control Protocol/Internet Protocol), the acknowledgment message is an essential part of the communication process, ensuring reliable data transmission. Upon receiving the acknowledgment message, the transfer device 100 can proceed with further actions, such as sending additional files 22 or initiating follow-up processes.

In one embodiment, the transfer device 100 may be configured to validate the files 22 transfer. Such validation may be performed using various methods such as acknowledgment tracking. Additionally or alternatively, the transfer device 100 may receive acknowledgment messages sent by the remote platform 200 to confirm successful reception of the files 22.

The remote platform 200 may further comprise a file parser 32, configured to extract relevant information from the received files 22. This parsing functionality enables the remote platform 200 to identify and isolate pertinent data points for inclusion in medical reports 36, saving physicians valuable time and effort in manual data extraction. For instance, the file parser may parse the filename, in order to specifically identifying information such as device identification data (like the medical device 10 type, the manufacturer name, the serial number and/or technical data (like the medical device 10 settings, the medical device 10 battery level) and/or measurements data (like last alerts messages, event logs, measurements).

Furthermore, the remote platform 200 may incorporates a data normalizer 33, which standardizes and structures the extracted relevant information to ensure consistency and accuracy across medical reports 36. Moreover, a common “multi-manufacturers” format may be used to harmonize the data coming from different manufacturers.

The remote platform 200 may integrate with a patient database 34, serving as a centralized repository of patient information. This patient database 34 enables physicians to access comprehensive patient histories, track treatment progress (for example monitor the evolution over time of the patient's cardiac condition), and generate personalized medical reports 36 tailored to individual patient needs.

The remote platform 200 may further comprise a medical report 36 generator (not represented), configured to automate the process of generating medical reports 36 based on the data contained in the files 22. This medical report generator may employ algorithms and templates to analyze the collected data, interpret trends, and compile comprehensive reports summarizing the patient's health status, and any notable observations or concerns.

Additionally, the remote platform 200 may further comprise an interface (not represented), accessible via a webpage or web-based application, providing physicians with access to the generated medical reports 36. Through this interface, physicians can review and validate the accuracy of the generated reports (for instance in a non-modifiable PDF format), for instance by adding a signature (numerical signature or image inserted). Furthermore, the interface may allow for modifications to be made on the medical reports 36 as necessary.

Embodiments of the remote platform 200 may vary, with possibilities including cloud-based solutions accessible via web browsers or alternatively internal systems deployed within healthcare facilities.

As mentioned above, the transfer device 100 is configured to allow a seamless transfer of the files 22 from the programming device 20, to the remote platform 200 described above.

As illustrated on FIG. 2 and FIG. 5, the transfer device 100 may comprise a housing 101. The housing 101 may have a length comprised between 5 cm and 15 cm, a width comprised between 3 cm and 10 cm and a height comprised between 1 cm and 3 cm.

The transfer device 100 may comprise on one of its walls at least one USB connection port 44 configured to be connected on at least one port of the programming device 20. The USB connection port 44 is configured to exchange data with the programming device 20.

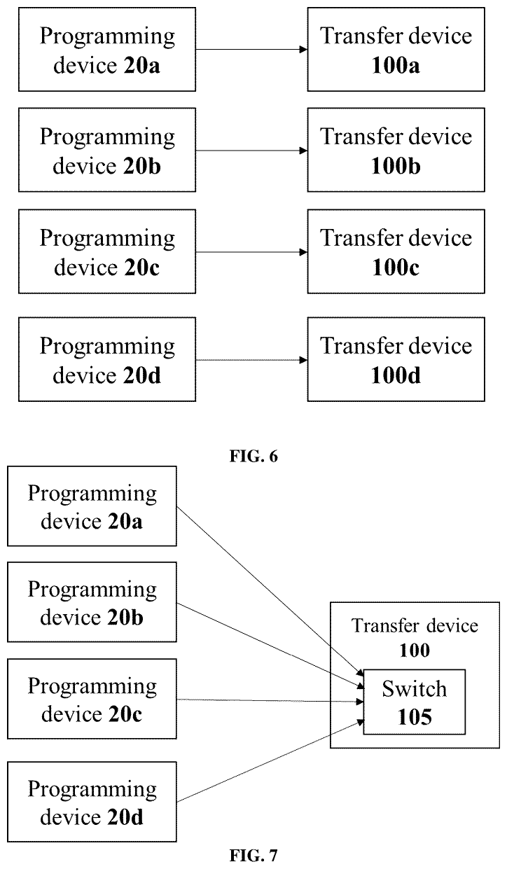

According to one embodiment, the transfer device 100 may be configured to be connected to only one programming device 20. In other words, as illustrated on FIG. 6, to each programming device 20a, 20b, 20c, 20d, a corresponding unique transfer device 100a, 100b, 100c, 100d is connected. Such connection may be made through a USB connection port 44 of the transfer device 100a, 100b, 100c, 100d.

Alternatively, the transfer device 100 may be configured to be connected to multiple programming devices 20.

According to a first example illustrated on FIG. 7, the transfer device 100 may comprise a switch 105. Such switch 105 may be internal to the transfer device 100 or external. The switch 105 may be an electronic switch that may comprise at least one transistor or a mechanical switch such as a toggle switch, a push-button switch, a rotary switch, or a membrane switch. The programming devices 20a, 20b, 20c, 20d may be configured to be connected to the switch, for example trough USB connection. The switch 105 may commute to select one programming device 20a, 20b, 20c, 20d among the multiple programming devices 20a, 20b, 20c, 20d so as to allow connection with the transfer device 100.

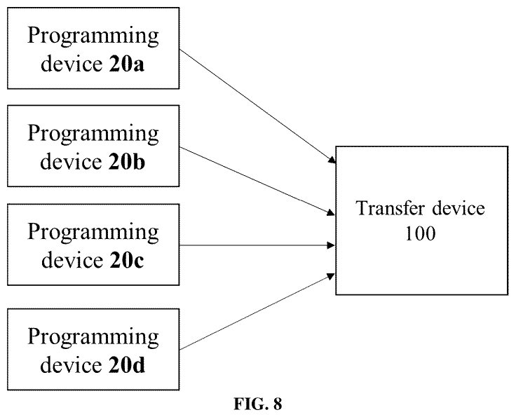

According to a second example, illustrated on FIG. 8, the transfer device 100 may comprise multiple USB ports, each USB port being configured to be connected to one programming device 20a, 20b, 20c, 20d. Additionally, the programming device 100 may be configured to detect which programming device 20a, 20b, 20c, 20d is switched on and running and to establish a connection with this programming device 20a, 20b, 20c, 20d.

Moreover, the transfer device 100 may include a power supply port 43 configured for connection to a power supply 51 that may be an external power supply. Alternatively or additionally, the transfer device 100 may comprise a rechargeable battery, that may allow the transfer device 100 to operate without connection to a power supply 51 for a certain amount of time. Alternatively or additionally the transfer device 100 may receive power via at least one of the USB ports.

The transfer device 100 may further comprise a memory 44 (RAM, GRAM or ROM type, or combination thereof). In one embodiment, the memory 44 is a RAM so that any file 22 contained in the RAM may be deleted when the transmission device 100 is powered off. This allows a better protection of patients' private data.

The transfer device 100 may also feature a USB service 46. The USB service 46 and the transfer device typically refers to a software component or functionality that enables communication and interaction between a USB-connected device (e.g. transfer device 100) and a host system (e.g. programming device 20). The USB service 46 may for instance be configured to implement protocols or drivers to manage the USB connection and enable data 12 transfer 11. For instance, the USB service 46 may be configured to share a memory space 47 with the programming device 20. To that end, memory allocation procedures may be implemented to reserve a region of the transfer device memory 44 for the memory space 47. Subsequently, the USB service is configured to implement functions that may enable the programming device 20 to access (reading and writing) the memory space 47. To that end, the transfer device 100 may be configured to emulate the behavior of a USB storage device. This may include functions such as USB enumeration, to respond to requests sent by the programming device 20, data transfer functions to facilitate efficient read and write requests, device identification function, to provide relevant information (such as vendor, product IDs, device capacity, and file system format) to the programming device 20, access control features to manage files 22 access permissions, and status indication capabilities to communicate the transfer device 100 status to the programming device 20.

In one embodiment, the USB service 46 may use “USB gadget mass storage” module from Linux kernel to emulate the behavior of a USB peripheral device to be connected to the USB port of the programming device 20. Alternatively, the transfer device 100 may comprise a microcontroller configured to implement a USB ode device such as for instance STM32 or ESP32.

During export of files 22 from the programming device 20, the USB service 46 may be further configured to detect activity from the programming device 20 (e.g. if export is still ongoing, by examining whether the amount of data in the memory space 47 is still changing) and/or to wait for an end of activity for a fixed or variable duration, for instance comprised between 1 second and 10 minutes, preferably 3 seconds.

Alternatively or additionally, the transfer device 100 may feature a Bluetooth service (not represented). The Bluetooth service may, for instance, be configured to implement protocols or drivers to manage the Bluetooth connection and enable files 22 transfer from the programming device 20. The Bluetooth service may be configured to implement functions enabling the programming device 20 to access (read and write) the memory space 47. To accomplish this, the transfer device 100 may emulate the behavior of a Bluetooth-enabled device, such as a printer. This could involve functions such as Bluetooth pairing, to establish a connection with the programming device 20, data transfer functions to facilitate efficient data exchange, device identification functions, access control features to manage file access permissions, and status indication capabilities to communicate the status of the transfer device 100 to the programming device 20.

After transfer of the files 22 from the programming device 20, the transfer device 100 may optionally be further configured to end sharing of the memory space 47 with the programming device 20.

Furthermore, the transfer device 100 may incorporate a transmission device 48 capable of managing communication and files 22 transfer with the remote platform 200. As mentioned above, the transmission device 48 may for instance utilize Application Programming Interfaces (APIs) to communicate with the remote platform 200, for instance when requesting transfer, receiving the unique and time-limited URL for data transmission, receiving acknowledgment, and verifying the completion of the transfer.

Additionally, the transfer device 100 may be configured to initiate several attempts of sending the files 22 to the remote platform 200. If transmission of the files 22 fails, the transfer device 100 may be configured to delete the files 22 from the memory space 47. In the same manner, when transmission of the files 22 has ended (e.g. transfer has been validated), the transfer device 100 may be configured to delete the files 22 from the memory space 47. Additionally, when the transfer device 100 is powered off, the memory space 47 may be deleted.

According to an embodiment, the memory space 47 may comprise an accessible space for writing by the programming device 20 and a buffer space for transfer toward the remote platform 200. Once writing of the files 22 by the programming device 20 is completed, the content of the accessible space is transferred into the buffer space. The files comprised in the buffer space are then sent to the remote platform 200.

A status display device 49, possibly in the form of indicator lights, may be included on the housing 101 of the transfer device 100, to provide visual feedback on the transfer device's operational status.

If the remote platform 200 is accessible via internet connection, the transfer device 100 may be equipped with internet connectivity capabilities. For instance, the transfer device 100 may be connected to internet wirelessly, for example via Wi-Fi, 4G, 5G. Alternatively, connection to internet may be established using a wired connection, such as an ethernet cable. To that end, the transfer device 100 may comprise an ethernet port 42.

In its automatic actions, the transfer device 100 may for example execute the following computer implemented method:
establishing a connection between said transfer device 100 and said programming device 20 (step 71),
detecting when said at least one file 22 is written into said memory space 47 (step 72),
transmitting said at least one file 22 from said memory space to said remote platform 200 (step 73).

1. A transfer device for transferring at least one file stored on a programming device to a remote platform, said programming device being configured to generate said at least one file from at least one data received from a medical device, said transfer device comprising:
at least one input interface configured to receive said at least one file from said programming device,
a memory comprising a memory space configured to receive said at least one file from said at least one input interface,
at least one processor configured to:
detect when a connection is established between said transfer device and said programming device,
detect when said at least one file is written into said memory space after said connection is established,
at least one output interface configured to send said at least one file to said remote platform, wherein said at least one processor is further configured to transmit said at least one file, after detection that said at least one file has been written into said memory space, from said memory space to said remote platform via said at least one output interface.

2. The transfer device according to claim 1, wherein said transfer device is configured to emulate a behavior of a Universal Serial Bus (USB) storage device so that when said connection is established between said transfer device and said programming device, said programming device authorizes said writing of said at least one file into said memory space.

3. The transfer device according to claim 1, wherein said transfer device is configured to emulate a behavior of a printer so that when said connection is established between said transfer device and said programming device, said programming device authorizes said writing of said at least one file into said memory space.

4. The transfer device according to claim 1, wherein when transmitting said at least one file from said memory space to said remote platform fails, said at least one processor is configured to initiate again at least one attempt of transmitting said at least one file from said memory space to said remote platform.

5. The transfer device according to claim 1, wherein when a number of failed attempts of transmitting said at least one file from said memory space to said remote platform exceeds a threshold, said at least one processor is configured to delete said at least one file from said memory space.

6. The transfer device according to claim 1, wherein the at least one processor is further configured to delete said at least one file from said memory space after said at least one file has been correctly transmitted to said remote platform.

7. The transfer device according to claim 1, wherein said transfer device is configured to establish a connection with multiple programming devices.

8. The transfer device according to claim 7, wherein said at least one processor is further configured to select one programming device among the multiple programming devices from which said at least one input interface is configured to receive said at least one file.

9. The transfer device according to claim 1, wherein said at least one processor is further configured, before transmission of said at least one file to said remote platform, to send a transmission request to a transmitter service of said remote platform and to notify a transmission success to said transmitter service.

10. A computer-implemented method for transferring at least one file from a programming device to a remote platform, said programming device being configured to generate said at least one file from at least one data received from a medical device, said method being implemented with the transfer device according to claim 1, said method comprising the steps of:
establishing a connection between said transfer device and said programming device,
detecting when said at least one file is written into said memory space,
transmitting said at least one file from said memory space to said remote platform.

11. The method according to claim 10, further comprising notifying to a user success or failure of said transmission.

12. The method according to claim 10, further comprising storing said at least one file into a storage space of said remote platform.

13. The method according to claim 10, further comprising processing, by a file parser service of said remote platform, said at least one data comprised in said at least one file.

14. The method according to claim 13, wherein said processing comprises extracting at least one data of interest from said at least one file and automatically generating a medical report based on at least said data of interest.

15. The method according to claim 14, wherein when said data of interest comprise new data generated by said medical device, said processing comprises extracting said new data from said at least one file and automatically updating said medical report based at least on said new data.

16. The method according to claim 14, further comprising reviewing and validating, by a physician, the medical report via said remote platform.
